(19) United States
(12) Patent Application Publication
(10) Pub. No.: US 2024/0066266 A1
(43) Pub. Date: Feb. 29, 2024

(54) Non-Integrated Vascular Access System with Stabilization

(71) Applicant: Becton, Dickinson and Company, Franklin Lakes, NJ (US)

(72) Inventors: Jonathan Karl Burkholz, Salt Lake City, UT (US); Serena Robinson Agrawal, Salt Lake City, UT (US); Jason Andrew Wine, Brea, CA (US); Dorien Gemma Butter, Millcreek, UT (US)

(21) Appl. No.: 18/238,595

(22) Filed: Aug. 28, 2023

Related U.S. Application Data
(60) Provisional application No. 63/401,919, filed on Aug. 29, 2022.

(51) Int. Cl. A61M 25/00

(52) U.S. Cl. CPC A61M 25/0097; A61M 2039/0258

(57) ABSTRACT

A non-integrated vascular access system includes a connector having a distal port configured to be coupled to a hub of a catheter, a proximal port, a first lumen extending between the distal and proximal ports and in fluid communication with a second lumen defined by the catheter when the distal port is coupled to the hub, a side port arranged in the connector between the proximal and distal couplers, with the side port configured to be in fluid communication with the first lumen, and a fluid conduit having a proximal end coupled to the side port and a distal end, with the fluid conduit in fluid communication with the side port and configured to extend distally from the connector. The system includes a stabilization portion connected to the connector and configured to contact a patient, and a stabilization anchor platform spaced from the connector and positioned along the fluid conduit.

### CROSS-REFERENCE TO RELATED APPLICATION

The present application claims priority to U.S. Provisional Application Ser. No. 63/401,919 entitled “Non-Integrated Vascular Access System with Stabilization” filed Aug. 29, 2022, the entire disclosure of which is hereby incorporated by reference in its entirety.

### BACKGROUND OF THE INVENTION

### Field of the Invention

The present disclosure relates to a non-integrated vascular access system with a stabilization anchor platform.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. Among these limitations is the potential for a VAD to be dislodged from the patient due to unexpected forces applied to an IV line or extension set connected to the device, or applied directly to the VAD. Accordingly, there is a need in the art for devices and systems that prevent dislodgement of the catheter.

In some instances, an extension set can be coupled to a VAD, to provide additional flexibility for medical professionals in terms of administering and/or withdrawing fluids from the VAD. There are times when current VADs, and consequently extension sets, are manipulated manually to enable blood draw; however, doing so in an uncontrolled way can increase incidence of dislodgement of the VAD from the vasculature, thus shortening the life of the catheter indwell. Accordingly, a need exists in the art for improved devices, systems, and methods of manipulating an indwelling catheter to free the catheter tip and allow for blood collection and fluid delivery therefrom or therethrough.

### SUMMARY OF THE INVENTION

In one aspect or embodiment, a non-integrated vascular access system includes a connector having a distal port configured to be coupled to a hub of a catheter, a proximal port, a first lumen extending between the distal port and the proximal port, with the lumen being in fluid communication with a second lumen defined by the catheter when the distal port is coupled to the hub, a side port arranged in the connector between the proximal coupler and the distal coupler, with the side port configured to be in fluid communication with the first lumen, and a fluid conduit having a proximal end coupled to the side port and a distal end, with the fluid conduit in fluid communication with the side port and configured to extend distally from the connector.

The system may also include a stabilization portion connected to the connector, with the stabilization portion configured to be in contact with a patient, and a stabilization anchor platform spaced from the connector and positioned along the fluid conduit.

The stabilization anchor platform may include an attachment member configured to secure the stabilization anchor platform to a skin surface of a patient. The attachment member may include an adhesive pad. The system may further include a clamp positioned along the fluid conduit, with the stabilization anchor platform positioned between the clamp and the distal end of the fluid conduit. The system may further include a clamp positioned along the fluid conduit, with the stabilization anchor platform positioned between the clamp and the side port of the connector. The stabilization anchor platform may be moveable along the fluid conduit.

The system may further include an access connector positioned at the distal end of the fluid conduit, where the access connector includes at least one of a luer connector and a needle-free connector.

The system may further include a force controlled release connector coupled to the distal end of the fluid conduit, with at least a portion of the force controlled release connector configured to separate from fluid conduit when a predetermined separation force is applied to the force controlled release connector. The force controlled release connector may include a disconnect connector and a release feature, with the release feature engaged with the disconnect connector and configured to be disengaged from the disconnect connector when a predetermined separation force is applied to the disconnect connector. The disconnect connector may include a valve member.

The disconnect connector may include a first line extending from the disconnect connector and a second line extending from the disconnect connector, with the first line including a first port and the second line including a second port. The first and second ports may each include a needle-free connector.

The system may include a catheter hub and a catheter received by the catheter hub, with the catheter hub coupled to the connector.

The system may also provide that the disconnect connector may be re-connected with the release feature after the release feature and the disconnect connector are disconnected.

Alternatively, the system may also provide that the disconnect connector is prevented from being reconnected with the release feature after the release feature and the disconnect connector are disconnected.

### BRIEF DESCRIPTION OF THE DRAWINGS

FIG. 1 is a perspective view of a prior art extension set;

FIG. 2 is a top view of a non-integrated vascular access system according to one aspect or embodiment of the present application;

FIG. 3 is a top view of a non-integrated vascular access system according to a further aspect or embodiment of the present application;

FIG. 4 is a perspective view of a non-integrated vascular access system according to a further aspect or embodiment of the present application;

FIG. 5 is a top view of a force controlled release connection according to one aspect or embodiment of the present application;

FIG. 6 is a cross-sectional view of a force controlled release connection according to a further aspect or embodiment of the present application; and

FIG. 7 is a perspective view of a non-integrated vascular access system according to a further aspect or embodiment of the present application.

Corresponding reference characters indicate corresponding parts throughout the several views. The exemplifications set out herein illustrate exemplary embodiments of the disclosure, and such exemplifications are not to be construed as limiting the scope of the disclosure in any manner.

### DETAILED DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a catheter insertion device in preparation for or during use. Similarly, in this application, the terms “in the distal direction” and “distally” mean in the direction toward the distal tip of the needle or catheter of the system, and the terms “in the proximal direction” and “proximally” mean in the direction opposite the direction of the distal tip of the needle or catheter.

Provided herein are devices and systems for securing and/or stabilizing extension sets used with indwelling catheters, such as peripheral intravenous catheters (PIVCs). While certain devices (e.g., blood draw devices) are discussed below in terms of devices that may be used with PIVCs and extension sets, as described herein, those of skill will appreciate that any number of different devices for introducing an instrument, including instruments ranging from tubes, probes, sensors, wiring, fiber optics, guidewires, etc., may be used within the scope of the present disclosure.

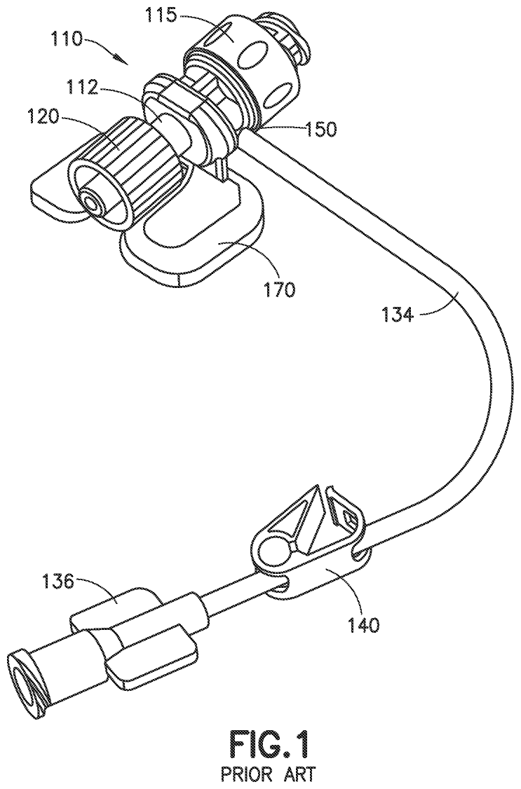

Referring now to FIG. 1, shown is a prior art extension set, including a stabilizing connector 110 and fluid conduit 134. The stabilizing connector 110 is configured to be placed in contact with the skin of a patient at or near an insertion site of an indwelling or placed vascular access device (VAD), such as those described above and known to those of skill in the art. The stabilizing connector 110 is configured to couple to and/or otherwise engage the VAD. Once coupled to a VAD, the stabilizing connector 110 can be secured to the skin of the patient (e.g., via medical tape, a clear sterile barrier such as Tegaderm™, and/or the like), which in turn secures and/or stabilizes at least a portion of the VAD relative to the patient, as described in further detail herein. The stabilizing connector 110 and related features may be the same or similar to extension set and connector shown and described in U.S. Patent Application Publication No. 2019/0160275, which is incorporated herein by reference in its entirety.

The stabilizing connector 110 can be any suitable shape, size, and/or configuration. In the illustrated non-limiting embodiment, the stabilizing connector 110 has a connector portion 112 and a stabilization portion 170. In some embodiments, the stabilizing connector 110 can be configured as a combination of one or more stabilization device(s) and an extension set. Each of the connector portion 112 and/or the stabilization portion 170 can be arranged in any suitable manner to facilitate at least one of the functions of providing stabilization to one or more devices (e.g., a VAD or the like) and/or at least one of the functions of providing an extension set for use with a VAD.

The connector portion 112 has a proximal port with a coupler 115, a distal port with a distal coupler 120, and defines at least one lumen (not shown) extending through or otherwise in fluid communication with the couplers 115 and 120. The proximal coupler 115 and/or the distal coupler 120 can be, for example, male or female luer locks and/or any other suitable coupler. The proximal coupler 115 can be physically and fluidically coupled to any suitable medical device such as those described above. The distal coupler 120 can be physically and fluidically coupled to, for example, a VAD or the like such that the lumen of the connector portion 112 is at least selectively in fluid communication with the VAD and/or a portion of the body in which the VAD is at least partially disposed. In some embodiments, the lumen of the connector portion 112 can be substantially straight and/or can allow for a substantially straight line of sight therethrough. In some embodiments, an inner surface of the connector portion 112 can be configured to provide alignment, guidance, centering, etc., to an object or device (e.g., a blood draw catheter or the like, as described above) being advanced therethrough.

In some embodiments, the couplers 115 and/or 120 can be arranged and/or configured to accept a click-to-connect coupling (e.g., a click-lock-Snap™ connection), a threaded coupling, a luer connection, and/or the like. In some non-limiting embodiments, the couplers 115 and/or 120 may include, for example, a spin collar or the like and/or may otherwise be configured to form relatively easy, secure, and fluid tight connections. In some embodiments, the couplers 115 and/or 120 can be a needleless or needle-free connector, an independent connector, and/or a swappable connector, and/or can be compatible with any suitable valve and/or seal (e.g., a valve used in a luer lock, a needle-free connector (NFC) (also known as a needless access connector, (NAC)), a split septum, and/or the like). As a specific, non-limiting example, the proximal port 115 can be configured as an NFC or the like and may include an NFC valve (e.g., similar to or the same as known needle-free connectors and/or known NFC valves). The coupler 115 is configured to provide catheter access to peripheral devices such as, e.g., a blood draw device (e.g., PIVO™ from Becton, Dickinson and Company), or a vascular access probe (VAP) for in-vein digital measurement of patient data such as temperature, pH, lactate, and/or other blood-based measurements.

In some embodiments, the distal coupler 120 can be configured to couple to, for example, a VAD such that engagement allows a tapered portion thereof to slip into a hub or the like to establish hemostasis, and can include a floating collar or the like such that the connector portion 112 remains coupled to the VAD during manipulation of one or more devices coupled thereto. In some embodiments, the couplers 115 and/or 120 can be configured to be compatible with any suitable known coupler or connector. In some embodiments, the distal coupler 120 can be or can include, for example, a floating male luer spin collar, a rotating male luer lock collar, or the like. Moreover, in some embodiments, the couplers 115 and/or 120 can include protective caps and/or the like that are removably coupled to the couplers 115 and 120. Such protective caps can be slip or friction fit or can be coupled via a threaded coupling.

The connector portion 112 also includes and/or defines one or more additional ports, such as a side port 150. Side port 150 can be included in and/or can be a part of the connector portion 112, the proximal coupler 115, the distal coupler 120, and/or a combination thereof. For example, side port 150 may be distal to the proximal coupler 115. In other embodiments, however, a connector portion 112 can include and/or define a side port 150 that is formed by a structure or feature forming a proximal coupler. In some instances, positioning side port 150 in a desired position along a length of the connector portion 112, between the couplers 115 and 120, can allow for a reduced length of the connector portion 112 and/or can facilitate flushing and/or fluid transfer via side port 150.

In some embodiments, the arrangement of side port 150 can be such that the connector portion 112 forms, for example, a Y-connector or a T-connector. More particularly, side port 150 can be disposed substantially perpendicular (e.g., about 90 degrees) to the lumen of the connector portion 112 and near or adjacent the proximal coupler 115. In non-limiting embodiments, side port 150 extends from connector portion 112 at an angle, relative to the lumen between distal coupler 120 and proximal coupler 115, that is not 90 degrees (e.g., side port 150 extends at an angle of, for example and without limitation, such as from 15-165 degrees, all values and subranges therebetween inclusive). In non-limiting embodiments, more than one side port 150 is included in connector portion 112.

In some embodiments, a position of the side port 150 can be shifted along the connector portion 112 in a desired manner to enable use of a shorter proximal connector (e.g., the proximal coupler 115). In some embodiments, the side port 150 and/or the fluid conduit 134 coupled thereto can be and/or can form at least a portion of a fluid line that can be used to deliver fluid, remove fluid, flush fluid, and/or the like. In such embodiments, for example, an arrangement in which side port 150 is disposed adjacent to the proximal coupler 115 can enable flushing of the proximal coupler 115, any valve(s) included therein, and/or a space between any valve(s) and an inner surface of the connector portion 112 (e.g., defining at least a portion of the lumen). In other embodiments, the connector portion 112 can be a single port connector that does not include the port 150 (and that can be flushed, for example, through the proximal port 115).

Side port 150 defines a lumen (not shown) that is in fluid communication with the lumen between distal coupler 120 and proximal coupler 115. In other words, the connector portion 112 and/or the side port 150 can include and/or define a first lumen (e.g., between distal coupler 120 and proximal coupler 115) and a second lumen (e.g., lumen defined by side port 150 and fluid conduit 134). As such, side port 150 can provide access to the lumen between distal coupler 120 and proximal coupler 115, which in turn can provide access to a device (e.g., a VAD) that is coupled to the distal coupler 120 and/or can provide access to a portion of the body in which the VAD is at least partially disposed.

As shown in FIG. 1, side port 150 may be coupled to fluid conduit 134 that is in fluid communication with the lumen of the side port 150. In some embodiments, the stabilizing connector 110 and/or any suitable portion thereof can include one or more features configured to manage and/or direct at least a portion of the fluid conduit 134 extending from side port 150. As shown, an end portion of the fluid conduit 134 (e.g., an end portion opposite side port 150) can include and/or can be coupled to an attachment device, coupler, connector, port, luer, and/or the like. For example, in the embodiment shown in FIG. 1, fluid conduit 134 includes a luer 136 at a distal end thereof, where the luer 136 may be configured to couple to any suitable device, such as a fluid source, a fluid collection device, an evacuated container, a pump, a syringe, and/or any other suitable device. Moreover, in some embodiments, the stabilizing connector 110 can include a clamp 140 coupled to the fluid conduit 134 and configured to selectively engage the fluid conduit 134 to constrict, crimp, clamp, and/or otherwise occlude a lumen defined by the fluid conduit 134 to limit and/or substantially prevent a flow of fluid therethrough. Although the clamp 140 is shown as being separate from luer 136, in other embodiments, at least a portion of the clamp 140 can be integrated into the luer 136.

In some embodiments, the stabilizing connector 110 can be configured for use with and/or configured to control a pressure or flow rate through at least a portion of the connector portion 112 and/or the lumen thereof. In some embodiments, the connector portion 112 can include and/or can accept one or more backflow preventers and/or valves (e.g., anti-reflux valves, check valves, split septums, and/or the like), one or more pressure regulators, and/or any other suitable flow control device.

The stabilization portion 170 is coupled to the connector portion 112 and is configured to be placed in contact with a portion of a patient (e.g., the skin of the patient) at or near an insertion site associated with the VAD (or other similar device). The stabilization portion 170 can be any suitable shape, size, and/or configuration. For example, in some embodiments, the stabilization portion 170 can be and/or can form a base structure that is angled, tapered, flared, curved, rounded, and/or the like. In some embodiments, the stabilization portion 170 can have a base surface (or bottom surface) that has a contour and/or shape that is generally concave. In some embodiments, the concave contour and/or shape of the base surface can be based at least in part on a curvature and/or shape of a portion of the patient's anatomy. In some embodiments, forming the contour and/or shape of the base surface to be similar to and/or at least partially based on the curvature and/or angle of an IV insertion site of a patient, for example, can increase a surface area of the stabilization portion 170 (e.g., base surface) that is in contact with the skin of the patient, which in turn can increase the stability of the stabilizing connector 110 and reduce a pressure associated with the stabilizing connector 110 when secured to the skin of the patient, as described in further detail herein.

In some embodiments, the base surface of the stabilization portion 170 may be selectively formed from one or more materials (e.g., a relatively hard material and/or a relatively soft material), configured to provide both stabilization and comfort. In some embodiments, the stabilization portion 170 can be configured to provide increased stabilization to a given or desired portion (e.g., a proximal portion, a distal portion, one or more side portions, and/or the like). In some embodiments, the stabilization portion 170 can be reconfigurable, which can allow a user to selectively control an amount of stabilization provided by the stabilization portion 170. Moreover, in some such embodiments, a user can reconfigure (e.g., bend, flex, deform, conform, stretch, break, cut, add to, etc.) one or more portions of the stabilization portion 170 to, for example, control an amount or manner of stabilization, to conform at least a portion of the stabilization portion 170 to the contours of a specific patient, to reduce or substantially prevent pressure points, and/or the like.

The base surface of stabilization portion 170 may include one or more contours, recesses, notches, cutouts, channels, etc., (referred to herein as a “recess”) configured to reduce an amount of force exerted by the stabilization portion 170 on or more veins and/or arteries of the patient (e.g., the vein in which the VAD is disposed), which might otherwise result in an occlusion of and/or a reduced flow rate through at least a portion of the vasculature. The contour and/or shape of the base surface can be based, for example, at least in part on a curvature and/or shape of a portion of the patient's anatomy. In some embodiments, at least one of the one or more recesses can substantially extend between a proximal edge and a distal edge of the base surface.

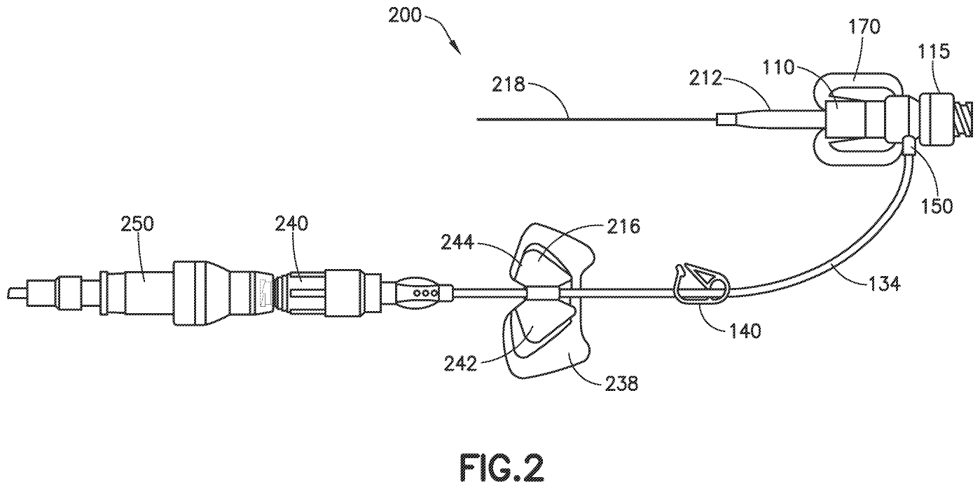

Referring to FIGS. 2-4, in one aspect or embodiment, a non-integrated vascular access system 200 includes the stabilizing connector 110, the stabilization portion 170 connected to the stabilizing connector 110, and a stabilization anchor platform 216 spaced from the stabilizing connector 110 and positioned along the fluid conduit 134 of the stabilizing connector 110. The system 200 may further include a catheter hub 212 and a catheter 218 received by the catheter hub 212, with the catheter hub 212 coupled to the stabilizing connector 110. In one aspect or embodiment, the catheter hub 212 is the AccuCath™ catheter system commercially available from Becton, Dickinson and Company.

The stabilization anchor platform 216 is configured to provide a securement point to a skin surface of a patient and minimize catheter dislodgement, infiltration, or catheter movement that may result in a tip of the catheter 218 from being pulled out of a vein or artery. The stabilization anchor platform 216 is configured to isolate external applied forces from the catheter hub 212 and insertion site to limit movement of the catheter 218 and/or dislodgement of the catheter 218 due to forces being applied to an intravenous line or extension sets. The system 200 is configured to provide sufficient compliance, flex, or slack between the stabilization anchor platform 216 and the catheter hub 212 to allow some displacement of the stabilization anchor platform 216 relative to the catheter 218 due to movement of the skin around the stabilization anchor platform 216 without applying excessive force and displacement of the catheter hub 212 itself. In some aspects or embodiments, the system 200 provides a high pressure rated catheter with blood draw and instrument delivery access and high pressure injection capabilities.

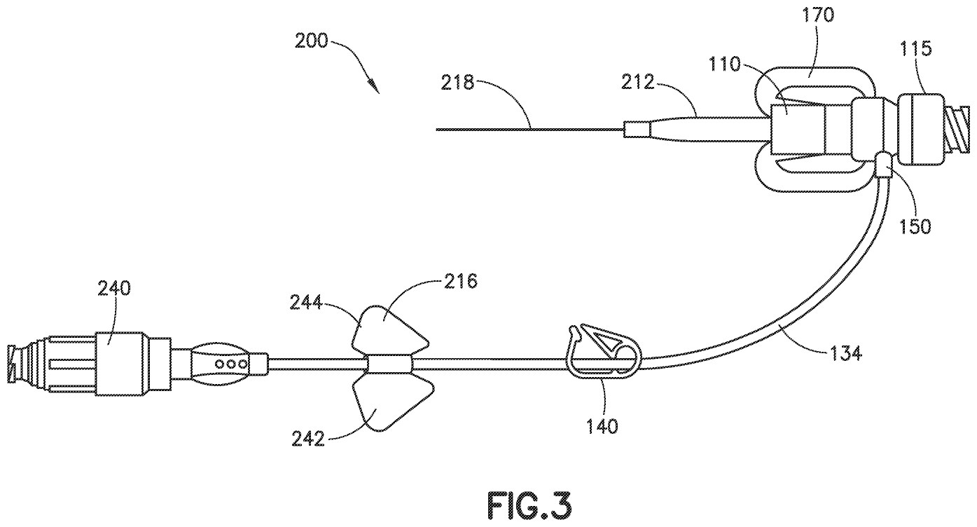

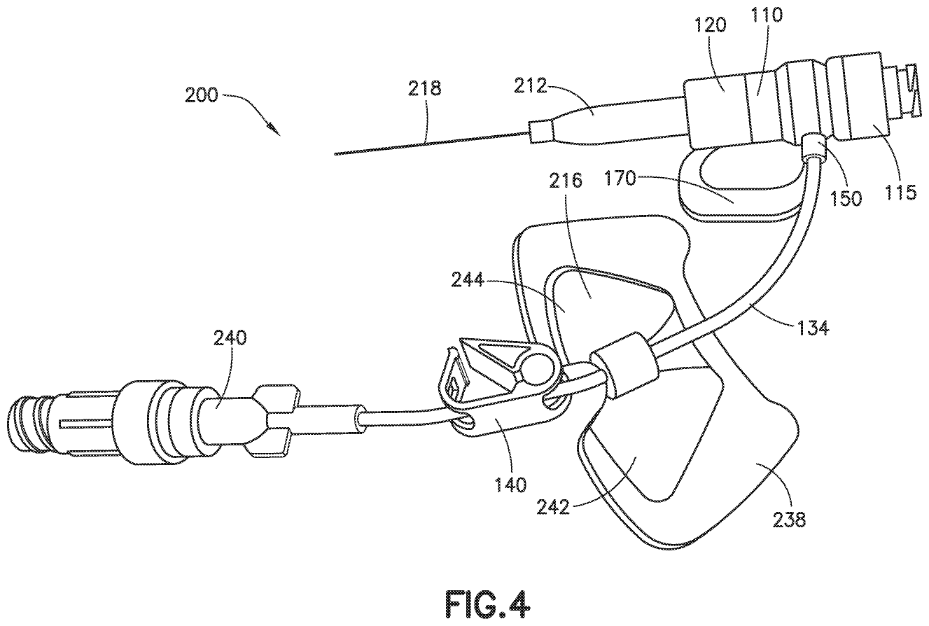

Referring again to FIGS. 2-4, in one aspect or embodiment, the stabilization anchor platform 216 includes an attachment member 238 configured to secure the stabilization anchor platform 216 to a skin surface of a patient. The attachment member 238 may include an adhesive pad. The attachment member 238 may be securement tape, an external stabilization device (ESD), dressing, and/or an anchoring platform adhesive with paper backing and skin adhesive. In one aspect or embodiment, the stabilization anchor platform 216 is shaped to fit within the StatLock™ ESD product from Becton, Dickinson and Co. As shown in FIG. 4, in some aspects or embodiments, the stabilization anchor platform 216 is positioned between the clamp 140 and the distal end of the fluid conduit 134. As shown in FIGS. 2 and 3, in some aspects or embodiments, the stabilization anchor platform 216 is positioned between the clamp 140 and the side port 150 of the stabilizing connector 110. The system 200 may further include an access connector 240 positioned at the distal end of the fluid conduit 134. The access connector 240 may include a luer connector and/or NFC. In some aspects or embodiments, the stabilization anchor platform 216 may also be integrated or coupled to the access connector 240.

The stabilization anchor platform 216 includes a pair of stabilizing wings 242, 244, although other suitable arrangements may be utilized. The stabilization anchor platform 216 may be fixed, removable, or moveable along the fluid conduit 134. The stabilization anchor platform 216 may be oval, triangular, round, square, rectangular, trapezoidal, or other suitable shape. The stabilization anchor platform 216 may be flexible, soft, semi-rigid, or rigid and may include features or a living hinge to promote flexibility for patient comfort. The stabilization anchor platform 216 may also include one or more features to improve attachment to the attachment member 238, such as protrusions, surface roughness, pockets, etc. The stabilization anchor platform 216 may include one or more colors or visual marking to provide catheter gauge, length, type, or procedure related information.

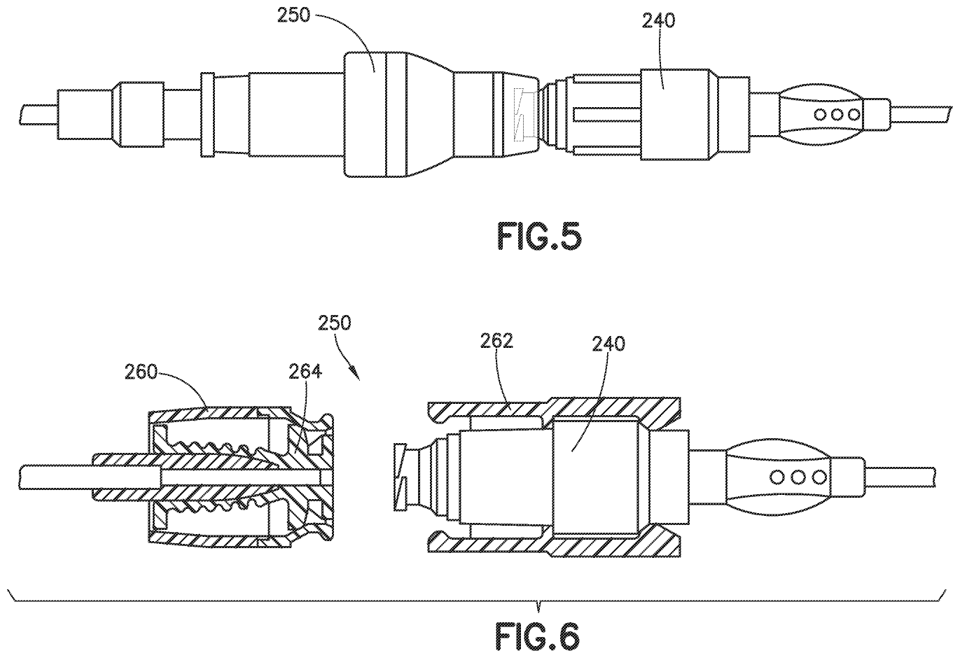

Referring to FIGS. 2 and 5-7, in some aspects or embodiments, the system 200 further includes a force controlled release connector 250 coupled to the distal end of the fluid conduit 134. At least a portion of the force controlled release connector 250 is configured to separate from the fluid conduit 134 when a predetermined separation force is applied to the force controlled release connector 250. At least a portion of the force controlled release connector 250 is configured to detach from the fluid conduit 134 to prevent excessive forces from being transmitted to the catheter hub 212 via the fluid conduit 134. For example, an intravenous line may be connected to the fluid conduit 134 via the force controlled release connector 250 such that excessive movement of the intravenous line will merely detach the connection between the fluid conduit 134 and the force controlled connector 250 rather than disrupting the placement of the catheter 218.

Referring to FIG. 6, the force controlled release connector 250 includes a disconnect connector 260 and a release feature 262 coupled to the access connector 240. The release feature 262 is engaged with the disconnect connector 260 and configured to be disengaged from the disconnect connector 260 when a predetermined separation force is applied to the disconnect connector 260 or the access connector 240. The disconnect connector 260 includes a valve member 264, which is configured to seal the disconnect connector 260 when separated from the access connector 240. In one aspect or embodiment, the release feature 262 is configured to be biased radially outward when the predetermined separate force is reached. The release feature 262 and the disconnect connector 260 may form a camming arrangement where an axial force causes the release feature 262 to move radially outward, thereby releasing the disconnect connector 260 from the release feature 262. The release feature 262 may be formed integrally with the access connector 240 or may be a separate component attached or coupled to the access connector 240.

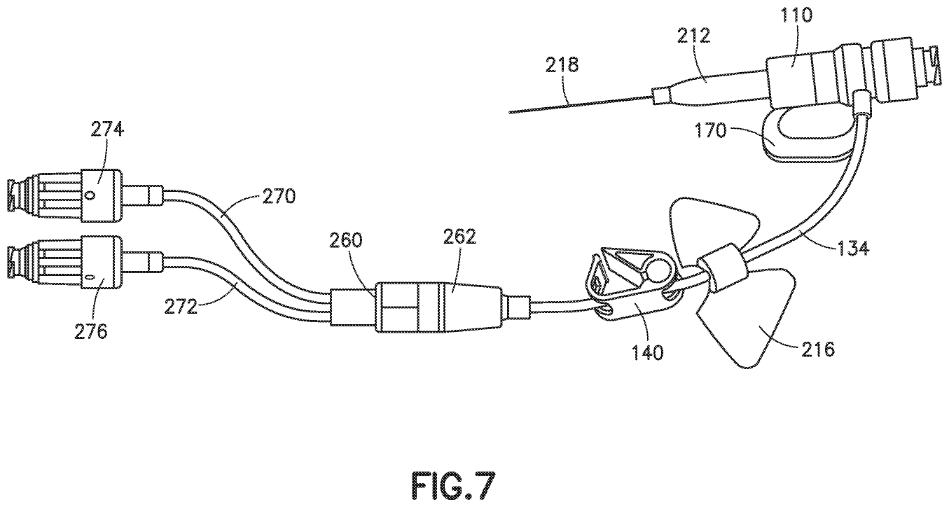

Referring to FIG. 7, in some aspects or embodiments, the disconnect connector 260 includes a first line 270 extending from the disconnect connector and a second line 272 extending from the disconnect connector 260. The first line 270 includes a first port 274 and the second line includes a second port 276. The first and second ports 274, 276 may each include a needle-free connector. In certain configurations, the disconnect connector 260 may be reconnected with the release feature 262, after the release feature 262 and the disconnect connector 260 are disconnected, such as once the predetermined separation force is no longer applied thereto. In other configurations, the disconnect connector 260 is prevented from being reconnected with the release feature 262 after the release feature 262 and the disconnect connector 260 are disconnected, thereby preventing reattachment.

While several embodiments were described in the foregoing detailed description, those skilled in the art may make modifications and alterations to these embodiments without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is:

1. A non-integrated vascular access system comprising:
a connector having:
a distal port configured to be coupled to a hub of a catheter;
a proximal port;
a first lumen extending between the distal port and the proximal port, the lumen being in fluid communication with a second lumen defined by the catheter when the distal port is coupled to the hub;
a side port arranged in the connector between the proximal coupler and the distal coupler, the side port configured to be in fluid communication with the first lumen; and
a fluid conduit having a proximal end coupled to the side port and a distal end, the fluid conduit in fluid communication with the side port and configured to extend distally from the connector;
and
a stabilization anchor platform spaced from the connector and positioned along the fluid conduit.

2. The system of claim 1, further comprising:
a stabilization portion connected to the connector, the stabilization portion configured to be in contact with a patient; and
a stabilization anchor platform spaced from the connector and positioned along the fluid conduit.

3. The system of claim 1, wherein the stabilization anchor platform comprises an attachment member configured to secure the stabilization anchor platform to a skin surface of a patient.

4. The system of claim 3, wherein the attachment member comprises an adhesive pad.

5. The system of claim 1, further comprising a clamp positioned along the fluid conduit, wherein the stabilization anchor platform is positioned between the clamp and the distal end of the fluid conduit.

6. The system of claim 1, further comprising a clamp positioned along the fluid conduit, wherein the stabilization anchor platform is positioned between the clamp and the side port of the connector.

7. The system of claim 1, wherein the stabilization anchor platform is moveable along the fluid conduit.

8. The system of claim 1, further comprising an access connector positioned at the distal end of the fluid conduit, wherein the access connector comprises at least one of a luer connector and a needle-free connector.

9. The system of claim 1, further comprising a force controlled release connector coupled to the distal end of the fluid conduit, wherein at least a portion of the force controlled release connector is configured to separate from fluid conduit when a predetermined separation force is applied to the force controlled release connector.

10. The system of claim 9, wherein the force controlled release connector comprises a disconnect connector and a release feature, the release feature is engaged with the disconnect connector and configured to be disengaged from the disconnect connector when a predetermined separation force is applied to the disconnect connector.

11. The system of claim 10, wherein the disconnect connector comprises a valve member.

12. The system of claim 10, wherein the disconnect connector comprises a first line extending from the disconnect connector and a second line extending from the disconnect connector, and wherein the first line comprises a first port and the second line comprises a second port.

13. The system of claim 12, wherein the first and second ports each comprises a needle-free connector.

14. The system of claim 1, further comprising a catheter hub and a catheter received by the catheter hub, wherein the catheter hub is coupled to the connector.

15. The system of claim 10, wherein the disconnect connector may be re-connected with the release feature after the release feature and the disconnect connector are disconnected.

16. The system of claim 10, wherein the disconnect connector is prevented from being reconnected with the release feature after the release feature and the disconnect connector are disconnected.
